(19) United States
(12) Patent Application Publication
(10) Pub. No.: US 2001/0019709 A1
(43) Pub. Date: Sep. 6, 2001

(54) USE OF METAL CHELATES AS RADIOSENSITIZERS

(76) Inventors: WERNER KRAUSE, BERLIN (DE); RUEDIGER LAWACZECK, BERLIN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/307,426

(22) Filed: May 7, 1999

(51) Int. Cl. A61K 31/00; A01N 61/00; A61M 36/14; A61K 51/00; C07C 1/00; A61K 49/00; A61B 5/055; A61K 49/04; A61B 8/00; A61B 5/00

(52) U.S. Cl. 424/1.65; 424/1.11; 424/9.1; 424/9.3; 424/9.4; 424/9.5; 424/9.6; 424/9.7; 534/7; 534/10; 540/474; 540/452; 534/16; 534/15; 534/14; 534/13; 534/12; 534/11

(57) ABSTRACT

The invention relates to compounds useful as radiosensitizers in tumor therapy.

### SUMMARY

The invention relates to compounds useful as radiosensitizers in tumor therapy.

This invention relates to novel radiosensitizing compounds, and in particular to metal-containing substances which are useful as radiosensitizers in tumor therapy.

### DESCRIPTION

### BACKGROUND OF THE INVENTION

Tumor therapy is presently based on three different approaches, namely chemotherapy, radiation therapy and surgery. Radiation therapy is often used as adjuvant or secondary treatment following surgical procedures to remove a tumor or in combination with chemotherapy. Accordingly, radiation therapy has a significant role in tumor therapy.

The search for drugs which enhance the cytotoxic activity of radiation (“radiosensitizers”) has been initiated early, and many different classes of compounds were studied for this purpose. Among them are nitroimidazoles (Misonidazol, Metronidazol, Etanidazol, Pimonidazol; J. Denekamp, Cancer Clin. Trials 1980, 3: 139-148; C. N. Coleman et al., Int. J. Radiat. Oncol. Biol. Phys., 1990, 18:389-93; T. S. Maughan et al., Int. J. Radiat. Oncol. Biol. Phys., 1990, 18:1151-6), 5-iododesoxyuridine (M. Deutsch et al., J. Natl. Cancer Inst. 1989, 81:1322-5), nicotinamide (G. G. Jonson et al., Radiother. Oncol. 1984, 1:349-53), cis-platin (M. Higi et al., Strahlentherapie 1982, 158:616-9) and other chemical structures.

Nitroimidazoles were especially effective, above all with hypoxic tumors. Some of these nitroimidazoles are currently in clinical trials. However, none of these drugs has so far been marketed. Thus, it is clear that a need exists for more potent radiosensitizing compounds which can be administered without toxic side effects.

### SUMMARY OF THE INVENTION

It was now surprisingly found that metal chelates are effective, as radiosensitizers. Additionally, it was found that these compounds not only are useful for therapy but also for diagnostic purposes so that with one single drug or even with one single injection of this drug, diagnosis and therapy of tumors is possible either simultaneously or consecutively.

### DETAILED DESCRIPTION OF THE PREFERRED EMBODIMENTS

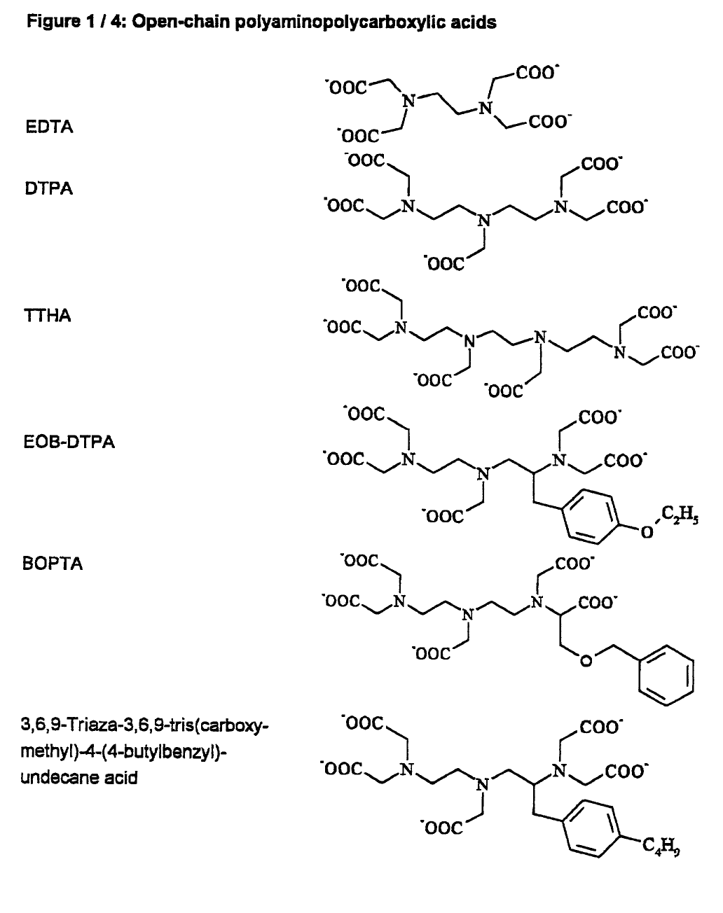

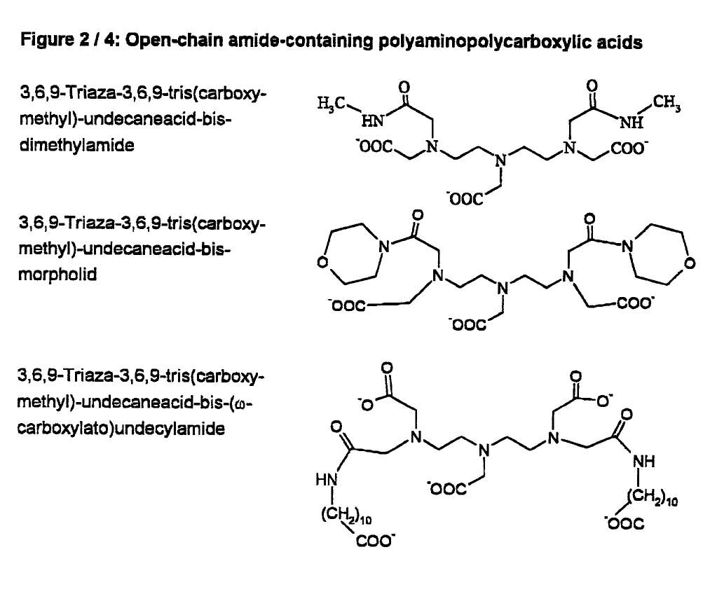

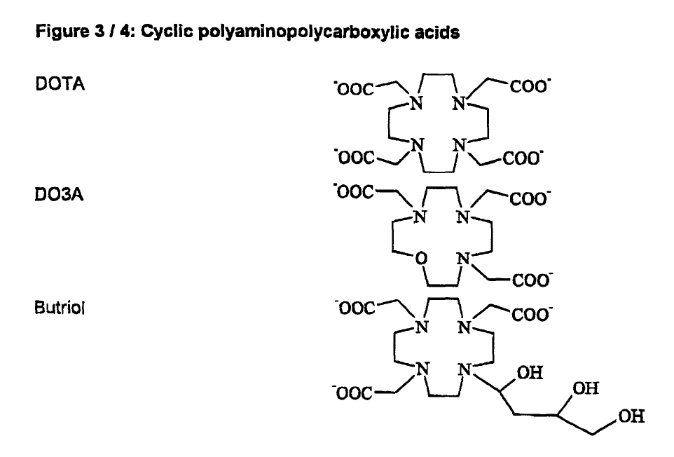

Metal chelates suitable as radiosensitizers are composed of a metal ion of atomic numbers 20-32, 39-51 or 57-83 and a polyaminopolycarboxylic acid as chelate forming ligand. Preferred ligands are open-chain polyaminopolycarboxylic acids such as EDTA, DTPA, EOB-DTPA, BOPTA, 3,6,9-triaza-3,6,9-tris(carboxymethyl)-undecane acid-bis-methylamide, 6,9-triaza-3,6,9-tris(carboxymethyl)-4-(4-butylbenzyl)-undecane acid, 6,9-triaza-3,6,9-tris(carboxymethyl)-4(4-butylbenzyl)-undecane acid bis(methylamide), 6,9-triaza-3,6,9-tris(carboxymethyl)-4-(4-butylbenzyl)-undecane acid bis(morpholid), 6,9-triaza-3,6,9-tris(carboxymethyl)-4-(4-butylbenzyl)-undecane acid bis(ωcarboxylato)-undecylamide (FIGS. 1 and 2) and cyclic polyaminopolycarboxylic acids such as DOTA, DO3A, Butriol (FIG. 3) or derivatives thereof. Additionally, the compounds might contain one or more cations of an organic or inorganic base or amino acid in order to compensate for the electric charge of the chelate.

Preferred compounds carry substituents which increase their lipophilicity. Suitable are alkyl, cycloalkyl, aryl, benzyl and phenyl moieties with up to 20 carbon atoms. These moieties might also contain hetero atoms such as oxygen, nitrogen or sulphur or combinations thereof, e.g. nitro groups such as found in nitroimidazoles. Suitable subsituents are also ethoxybenzyl, butylbenzyl, hexyl or benzyloxybenzyl moieties. The lipophilic substituents might also be amides of a carboxylic acid, e.g. undecylamide, butylbenzylamide or morpholid.

The new compounds are surprisingly able to reach hypoxic and necrotic tissue and enhance the cytotoxic activity of radiation. Tumors, especially hypoxic tumors can be treated very effectively by this procedure. Additionally, it is possible to simultaneously visualize tumors before, during and after therapeutic radiation by diagnostic imaging. This visualization is especially usefull for the exact location of the tumor in order to plan and control stereotactic irradiation. Using this procedure, the radiation beam can be focused exactly onto the tumor so that normal tissue is widely excluded from radiation damage.

Synthesis of the compounds

The synthesis of the compounds is well known to those skilled in the art and has been described in several publications (Weinmann HJ et al., Am, J. Roentgenol, 1984, 142:619, EP 71564, DE 3324236, DE 3324235, DE 3625417, EP 263059, DE 3710730, EP 450742, EP 448191, EP413405, EP 405704). The invention therefore relates to a method of using the compounds described in these publications as radiosensitizers.

Advantages of the compounds

Due to their metal ions, the compounds described are also suitable for diagnostic imaging. The great advantage of this class of substances is the possibility of combining diagnosis and therapy using one single drug and even one single injection of this agent. During diagnosis, using low-energy radiation, the drug is therapeutically inactive and of high tolerability. Only during therapy, i.e. by using radiation with higher energy, the drug is “switched on” and becomes toxic.

“Target-directed” (stereotactic) irradiation of tumors is state of the art. However, the exact localization of the tumor still constitutes a considerable problem. Normally, the localization is performed prior to radiation therapy, and some “land marks” are used for focusing the radiation beam. However, an exact localization of the tumor is not possible during the radiation therapy. The new agents allow for the simultaneous diagnosis and therapy of the tumor with one single drug and even one single injection so that the tumor can be localized at each moment of the therapy. This means that now “target-directed” radiosensitized destruction of tumors with increased efficacy and concomitant imaging of the tumor is possible. An additional feature is that the tumor can be localized now by diagnostic imaging prior to therapy, the therapeutic irradiation process will be started only after exact localization, maximal enrichment of the imaging agent/radiosensitizer and optimization of stereotactics.

The compounds described are suitable for the following imaging techniques:

1. for MRI as complexes of two and three-valid ions of elements with atomic numbers is 21-29, 42, 44 and 57-70. Suitable ions are for example chromium(III), iron(II), cobalt(II), nickel(II), copper(II), praseodym(III), neodym(III), samarium(III), and ytterbium(III) ions. Due to their high magnetic moments, gadolinium(III), terbium(III), dysprosium(III), holmium(III), erbium(III), manganese(II) and iron(III) ions are preferred.

2. for X-ray techniques as complexes of elements with high atomic numbers exhibiting sufficient absorption of X-rays. It was found that chelates containing an element of atomic numbers 57-83 as central ion are preferred for this technique.

3. for nuclear medicine techniques as chelates with radioactive central ions. Suitable are for example radioisotopes of the elements copper, cobalt, gallium, germanium, yttrium, holmium, lutetium, scandium, iron, europium, technetium, indium, ytterbium, gadolinium, samarium and iridium.

To further illustrate and explain the invention, several examples are presented below.

### EXAMPLE 1

Histiocytic lymphoma cells of human origin (U937) were used under normal conditions up to a cell densitiy of 600,000 to 700,000 cells/ml. At the time of the experiment (irradiation), the cell suspension was diluted with culture medium containing the test compound. The incubation medium without test compound was used as control. The test compound in this example was the bis(meglumine) salt of the gadolinium complex of DTPA (FIG. 1).

Following a 1 h-incubation period at 37° C. in the dark, a constant volume was transferred to a 6-well plate and irradiated at room temperature. Irradiation was performed with an RT 250 Müllerb/Philips apparatus using the following conditions: 180 kV, 15 mA, 0,5 mm Cu filter, FHA 30 cm, tubus 10×15 cm². The radiation dose was 4 Gray. After the irradiation and a total incubation time of 1 h, the cells were washed twice by centrifugation and resuspension in culture medium and transferred to culture flasks (T25) to a final volume of 5 ml of medium (20,000 cells/ml). Thereafter the cells were kept at 37° C. in the dark. The determination of cell density and cell volume was performed during a time period of 14 d using a CASY cell counting system.

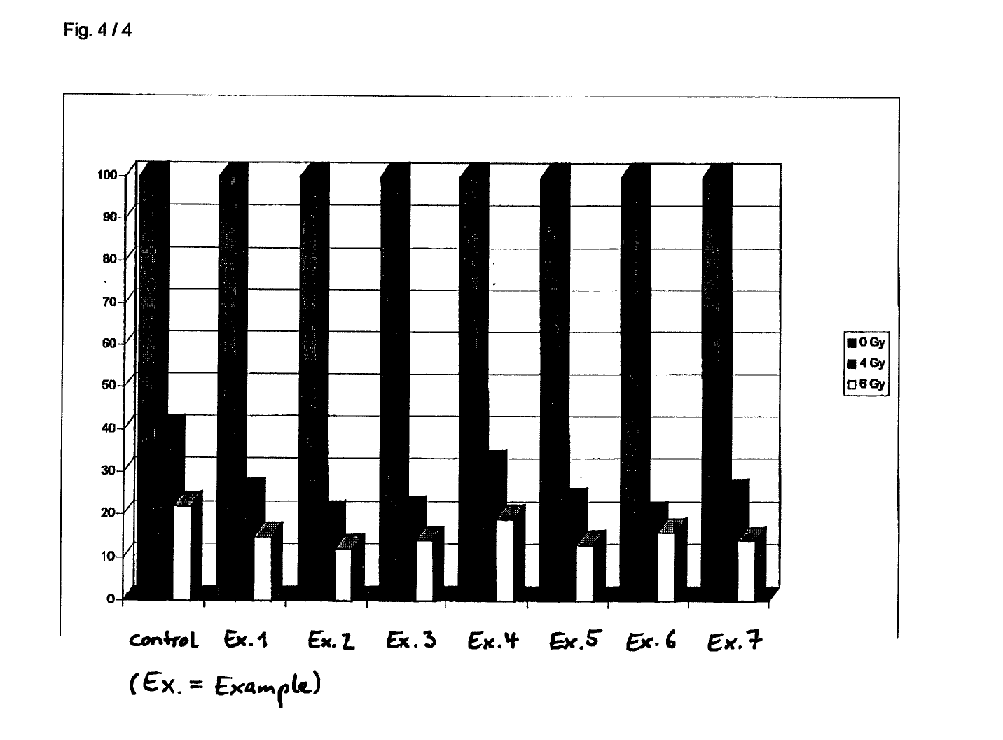

Non-irradiated cells showed a cell-number doubling rate of approx. 24 h. Normally, controls reach a cell density of 1,000,000 cells/ml after approx. 150 to 200 h. Due to the closed system, nutritives are consumed at this time point and further cell division is not observed. Irradiated cells reach this cell density (1,000,000 cells/ml) significantly later or even never-depending on the efficacy of the radiosensitizer. FIG. 4 summarizes the counting results after 150 h for a cell culture without (control) and with radiosensitizer as a function of the radiation dose (0, 4 und 6 Gray).

From the proliferation kinetics the growth rate can be determined as a function of treatment either graphically or by computer fitting. As a measure for efficacy, the cell density following a definite period of time might alternatively be used. Also, the cell density after a definite period of time relative to that of a non-irradiated control might be used as a measure for efficacy.

### EXAMPLE 2

The experiment was performed analogous to that described in example 1. The gadolinium complex of Butriol was used as radiosensitizer (FIG. 3).

The results are summarized in FIG. 4.

### EXAMPLE 3

The experiment was performed analogous to that described in example 1. The gadolinium complex of DTPA-bismorpholid was used as radiosensitizer (FIG. 2).

The results are summarized in FIG. 4.

### EXAMPLE 4

The experiment was performed analogous to that described in example 1. The gadolinium complex of 3,6,9-triaza-3,6,9-tris(carboxymethyl)-undecane acid-bis(ω-carboxylato)-undecylamide was used as radiosensitizer (FIG. 2).

The results are summarized in FIG. 4.

The experiment was performed analogous to that described in example 1. The gadolinium complex of Butriol encapsulated in liposomes was used as radiosensitizer (FIG. 3).

The results are summarized in FIG. 4.

### EXAMPLE 6

The experiment was performed analogous to that described in example 1. The gadolinium complex of EOB-DTPA was used as radiosensitizer (FIG. 1).

The results are summarized in FIG. 4.

### EXAMPLE 7

The experiment was performed analogous to that described in example 1. The ytterbium complex of EOB-DTPA was used as radiosensitizer (FIG. 1).

The results are summarized in FIG. 4.

1. A method of using metal chelates for the preparation of pharmaceutical compositions suitable for tumor therapy comprising metal chelates with metal ions of atomic numbers 20-32, 39-51 or 57-83 and polyaminopolycarboxylic acids as chelate forming ligand.

2. A method of using metal chelates for the preparation of pharmaceutical compositions suitable for radiation therapy of hypoxic tumors comprising metal chelates with metal ions of atomic numbers 20-32, 39-51 or 57-83 and polyaminopolycarboxylic acids as chelate forming ligand.

3. A method according to claims 1 and 2 comprising FDTA, DTPA, EOB-DTPA, BOPTA, 3,6,9-triaza-3,6,9-tris(carboxymethyl)-undecaneacid-bis-methylamide, 3,6,9-triaza-3,6,9-tris(carboxymethyl)-4-(4-butylbenzyl)-undecaneacid, 3,6,9-triaza-3,6,9-tris(carboxymethyl)-undecaneacid-bis-methylamide, 3,6,9-triaza-3,6,9-tris(carboxymethyl)-undecaneacid-bis-morpholid, 3,6,9-triaza-3,6,9-tris(carboxymethyl)-undecaneacid-bis-(ω-carboxylato)undecylamide as polyaminopolycarboxylic acid or a substituted derivative thereof.

4. A method according to claims 1 and 2 comprising DOTA, DO3A, Butriol or a substituted derivative thereof as polyaminopolycarboxylic acid.

5. A method according to claims 1 and 2 comprising a polyaminopolycarboxylic acid with at least one lipophilic substituent.

6. A pharmaceutical composition according to claims 1 and 2 for the use as radiosensitizer for tumor therapy.

7. A pharmaceutical composition for radiosensitizing hypoxic cells characterized in that a radiosensitizing amount of a compound according to the compounds of claim 1 is used in admixture with a pharmaceutically acceptable carrier.

8. A method of using metal chelates for the diagnosis and/or therapy of hypoxia comprising a metal chelate with metal ions of atomic numbers 20-32, 39-51 or 57-83 and a polyaminopolycarboxylic acid as chelate forming ligand.

9. A method of using metal chelates for the diagnosis and/or therapy of hypoxia tumors comprising a metal chelate with metal ions of atomic numbers 20-32, 39-51 or 57-83 and a polyaminopolycarboxylic acid as chelate forming ligand.

10. A method of using metal chelates for the diagnosis and/or therapy of ischemia comprising a metal chelate with metal ions of atomic numbers 20-32, 39-51 or 57-83 and a polyaminopolycarboxylic acid as chelate forming ligand.

11. A method of using metal chelates for the diagnosis and/or therapy of necrosis comprising a metal chelate with metal ions of atomic numbers 20-32, 39-51 or 57-83 and a polyaminopolycarboxylic acid as chelate forming ligand.

12. A method of using metal chelates for the simultaneous or subsequent diagnosis and therapy of tumors comprising a metal chelate with metal ions of atomic numbers 20-32, 39-51 or 57-83 and a polyaminopolycarboxylic acid as chelate forming ligand.

13. A method of using metal chelates according to previous claims comprising the chelators EDTA, DTPA, EOB-DTPA, BOPTA, 3,6,9-triaza-3,6,9-tris(carboxymethyl)-undecaneacid-bis-methylamide, 3,6,9-triaza-3,6,9-tris(carboxymethyl)-4-(4-butylbenyl)-undecaneacid, 3,6,9-triaza-3,6,9-tris(carboxymethyl)-undecaneacid-bis-methytamide, 3,6,9-triaza-3,6,9-tris(carboxymethyl)-undecaneacid-bis-morpholid, 3,6,9triaza-3,6,9-tris(carboxymethyl)-undecaneacid-bis-(ω-carboxylato)undecylamide as polyaminopolycarboxylic acid or a substituted derivative thereof.

13. A method of using metal chelates according to previous claims comprising the chelators DOTA, DO3A, Butriol or a substituted derivative thereof as polyaminopolycarboxylic acid.

14. A method of using metal chelates according to previous claims comprising a polyaminopolycarboxylic acid with at least one lipophilic substituent.

15. A method of using metal chelates according to previous claims for the preparation of diagnostics and therapeutics.
